(19) United States
(12) Patent Application Publication
(10) Pub. No.: US 2026/0337225 A1
(43) Pub. Date: Oct. 29, 2026

(54) SMALL PEPTIDE AND USE THEREOF IN MUCOSAL REPAIR

(71) Applicant: FORREST BIO-PHARM (SHENZHEN) CO., LTD., Shenzhen (CN)

(72) Inventors: Xitong ZHANG, Shenzhen (CN); Xiaojian YIN, Shenzhen (CN)

(21) Appl. No.: 19/115,301

(22) PCT Filed: Sep. 26, 2023
(86) PCT No.: PCT/CN2023/121316

§ 371 Date: Apr. 28, 2025

(30) Foreign Application Priority Data
Sep. 26, 2022 (CN) 202211172690.X

(51) Int. Cl. C07K 7/06; A61K 38/00; A61P 1/04; C07K 1/04; C07K 1/06

(52) U.S. Cl. CPC C07K 7/06; A61P 1/04; C07K 1/042; C07K 1/063; A61K 38/00

(57) ABSTRACT

Provided is a small peptide and use of the small peptide in mucosal repair, which belongs to the field of natural active ingredients. The small peptide is selected from the basic small peptide shown in SEQ ID NO.1 or SEQ ID NO.2, or a precursor small peptide comprising the basic small peptide, wherein the precursor small peptide can be enzymatically hydrolyzed by digestive enzymes in animals to obtain the basic small peptide. The small peptide exhibits mucosal repair efficacy. These peptides are synthesized in large quantities using Fmoc solid-phase synthesis method, and their properties related to mucosal repair are verified through animal experiments. Additionally, toxicological experiments confirm such small peptides lack of hepatotoxicity or nephrotoxicity, demonstrating high safety. Therefore, the developed small peptide possesses excellent druggability and can be widely applied in pharmaceuticals for gastrointestinal diseases, mucosal repair, and related therapeutic areas.

### CROSS-REFERENCE TO RELATED APPLICATIONS

This application claims priority to PCT Application No. PCT/CN2023/121316, having a filing date of Sep. 26, 2034, which is based on Chinese Application No. 202211172690.X, having a filing date of Sep. 26, 2022, the entire contents both of which are hereby incorporated by reference.

### SEQUENCE LISTING

This application contains a sequence listing, which is incorporated herein by reference in ST.26 XML format entitled “20250428 FP25USI234 43910 50005”, created Apr. 28, 2025, and is 2,977 bytes in size. The sequences contained in the sequence listing are found throughout the originally filed application and are referenced by the Specification.

### FIELD OF TECHNOLOGY

The following relates to the field of natural active ingredients, particularly it relates to a small peptide and use of the small peptide in mucosal repair.

### BACKGROUND

Digestive tract diseases, especially gastrointestinal diseases, are among the most common diseases today. The majority of digestive tract diseases are related to mucosal injuries, including oral ulcers, esophageal ulcers, gastric ulcers, intestinal ulcers, and genital mucosal inflammation.

Currently, conventional treatments for mucosal injuries in the digestive tract or other parts such as the oral cavity include acid suppression therapy and mucosal protective agents. Common medicament includes a proton pump inhibitor and an anti-inflammatory mouthwash, which primarily alleviate ulcer symptoms by inhibiting gastric acid or forming a physical protective layer on the mucosal surface, rather than directly applying to mucosal repair. Meanwhile, long-term use of acid suppressants may reduce the digestive ability of patients, potentially leading to malnutrition, digestive tract inflammation, diarrhea, and other adverse effects.

### SUMMARY

Based on this, it is necessary to address the above-mentioned problem and an aspect relates to a small peptide. Such small peptide is a small molecular polypeptide with excellent oral absorbability and bioavailability, mucosal repair effects, without hepatotoxicity or nephrotoxicity as proven by toxicological experiments, demonstrating high safety.

The small peptide is selected from the basic small peptides shown in SEQ ID NO.1 or SEQ ID NO.2, or a precursor small peptide comprising the basic small peptide, wherein the precursor small peptide can be enzymatically hydrolyzed by digestive enzymes in animals to obtain a basic small peptide.

During preliminary experiments, the inventors discovered that Polimi-1 rice variety (Guangdong Polimi Biotechnology Co., Ltd., Plant Variety Right No.: CNA20181039.9) had a lower protein absorption ratio compared to ordinary rice varieties. Clinically, patients with metabolic syndrome can consume this rice. During clinical observations (Approved by Medical Ethics Committee of Guangzhou Red Cross Hospital, N O. 2017-036-02), it was found that patients consuming such rice exhibited improved gastrointestinal function. Further analysis confirmed that the active component was a rice protein. By screening the method for extracting the rice protein, active mixture polypeptides, a rice polypeptide mixture, can be obtained by extraction.

Further, the inventors observed active monomers in the polypeptide mixture from Polimi-1 rice variety by mass spectrometry analysis of the polypeptides from various ordinary rice varieties and Polimi-1 rice variety. The small peptides identical to ordinary rice varieties and with high levels in ordinary rice varieties were excluded by a method of exclusion. Finally, the small peptides with higher levels in Polimi-1 rice variety and lower levels in other ordinary rice varieties were selected for further study, as well as those with higher levels in Polimi-1 rice variety and absent in other ordinary rice varieties. Since peptides with molecular weights below 1500 Daltons were extracted from the mixed polypeptide mixture previously obtained, other peptides above this threshold were excluded. Therefore, multiple candidate small peptides were recognized, and after repeated experiments and screening, the small peptide P1 shown in SEQ ID NO.1 and the small peptide P2 shown in SEQ ID NO.2 were found to have mucosal repair effects. These peptides were synthesized in large quantities using Fmoc solid-phase synthesis method, and their properties related to mucosal repair were verified through animal experiments. Meanwhile, toxicological experiments confirmed such small peptides lack of hepatotoxicity or nephrotoxicity, demonstrating high safety.

Another aspect relates to a method for preparing the small peptide, comprising Fmoc solid-phase synthesis method.

In one embodiment, the method comprises the following steps: covalently coupling the first amino acid at the C-terminus of a small peptide onto a solid-phase vehicle, then performing a synthesis from the N-terminus of this amino acid as a starting point, sequentially removing the amino-protecting group and reacting with an excessive activated second amino acid to extend a peptide chain, repeating above process to obtain the small peptide, cleaving the small peptide from the solid-phase vehicle, and purifying the small peptide to obtain a final product.

In one embodiment, the solid-phase vehicle is 2-chlorotrityl chloride resin.

The present disclosure also discloses use of the small peptide in the preparation of a medicament for preventing and treating a mucosal injury disease.

In one embodiment, the mucosal injury disease comprises oral ulcers, esophageal ulcers, gastric ulcers, intestinal ulcers, and genital mucosal inflammation.

In one embodiment, the gastric ulcers comprise acute gastric ulcers and chronic gastric ulcers.

Another aspect relates to use of the small peptide in the preparation of a medicament for preventing and treating gastrointestinal diseases.

In one embodiment, the gastrointestinal diseases comprise constipation, diarrhea, and gastric discomfort.

In one embodiment, the gastric discomfort comprises acid reflux, belching, indigestion, stomach bloating, stomach pain, vomiting, and nausea.

The present disclosure also discloses a pharmaceutical composition comprising the small peptide and a pharmaceutically acceptable excipient.

Compared to the prior art, the present disclosure has the following advantages:

The small peptides of the present disclosure are developed based on the discovery of a rice polypeptide mixture with mucosal repair effects. Through mass spectrometry peptide analysis comparing ordinary rice polypeptides versus Polimi-1 rice polypeptides, comparative peptide profiles are identified. After repeatedly performing experiments and screening, it is observed that the small peptide P1 (shown in SEQ ID NO.1) and small peptide P2 (shown in SEQ ID NO.2) exhibit mucosal repair effects. These peptides are synthesized in large quantities using Fmoc solid-phase synthesis method, and their properties related to mucosal repair are verified through animal experiments. Additionally, toxicological experiments confirm they lack of hepatotoxicity or nephrotoxicity, demonstrating high safety. Therefore, the small peptides of the present disclosure exhibit excellent drug potential and can be widely used in medicament for gastrointestinal diseases and mucosal repair.

Currently, observed indications comprise: constipation, diarrhea, gastric discomfort (acid reflux, belching, indigestion, stomach bloating, stomach pain, vomiting, nausea), mucosal injuries (oral ulcers, esophageal ulcers, gastric ulcers, intestinal ulcers), and loss of appetite (a significant increase in appetite and hunger sensation within 30 minutes after administration of the small peptides of the present disclosure). There are also a few reports (fewer than 10 cases) showing significant inhibitory and repair effects on male and female genital mucosal inflammation, as well as preventive effects on the recurrence of inflammatory bowel disease (IBD).

### BRIEF DESCRIPTION

Some of examples will be described in detail, with reference to the flowing figures, wherein like designations denote like members, wherein:

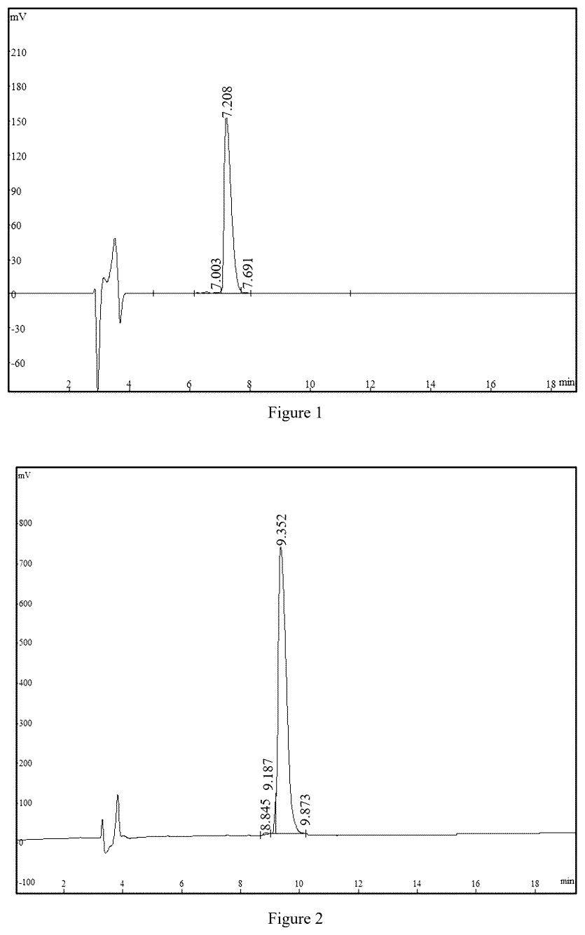

FIG. 1 depicts High Performance Liquid Chromatography (HPLC) chromatogram of the small peptide P1 of Embodiment 2;

FIG. 2 depicts HPLC chromatogram of the small peptide P2 of Embodiment 2;

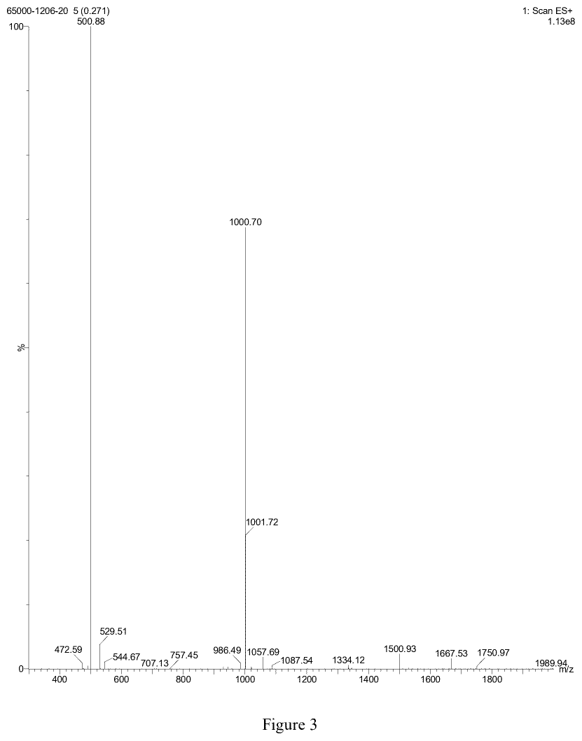

FIG. 3 depicts Mass spectrum of the small peptide P1 of Embodiment 2;

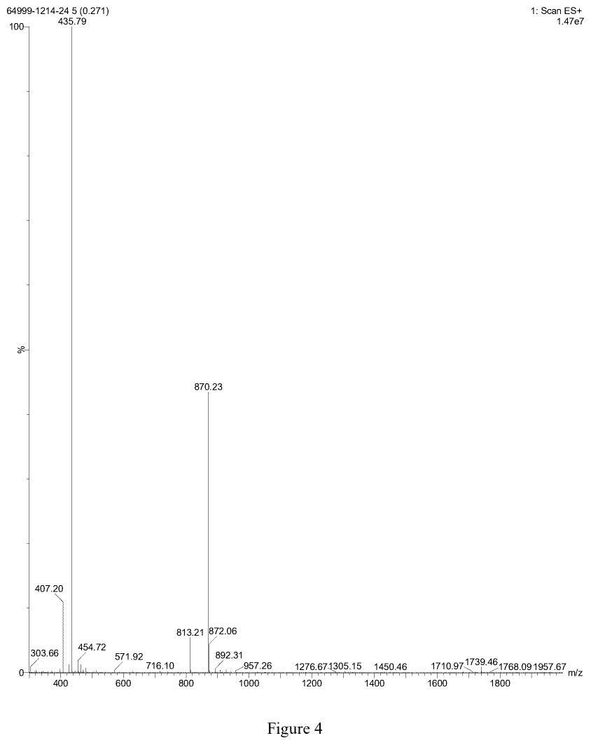

FIG. 4 depicts Mass spectrum of the small peptide P2 of Embodiment 2;

FIG. 5 depicts gastric tissue section images from the validation experiments of mouse model for acute gastric ulcer of Embodiment 3;

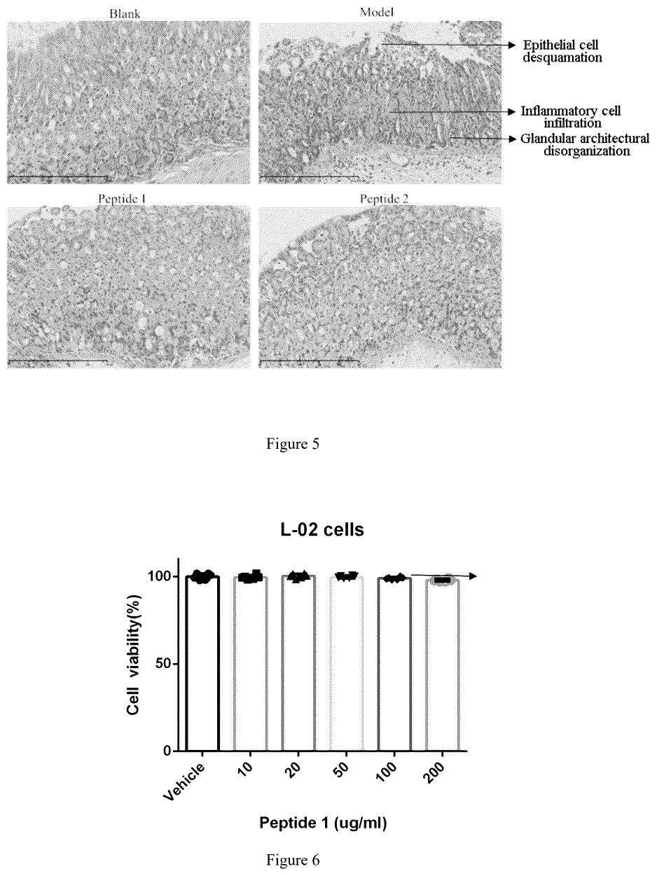

FIG. 6 depicts hepatotoxicity experimental results of the small peptide P1 of Embodiment 5;

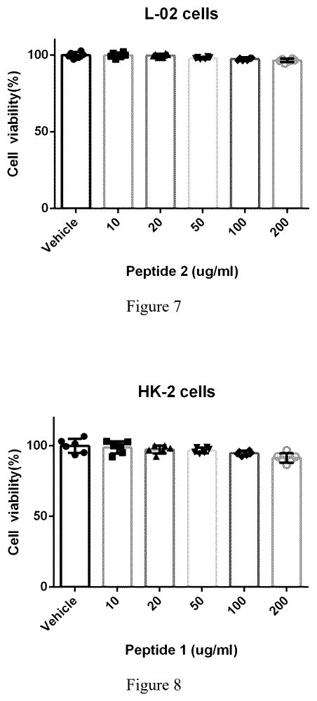

FIG. 7 depicts hepatotoxicity experimental results of the small peptide P2 in Embodiment 5;

FIG. 8 depicts nephrotoxicity experimental results of the small peptide P1 in Embodiment 5;

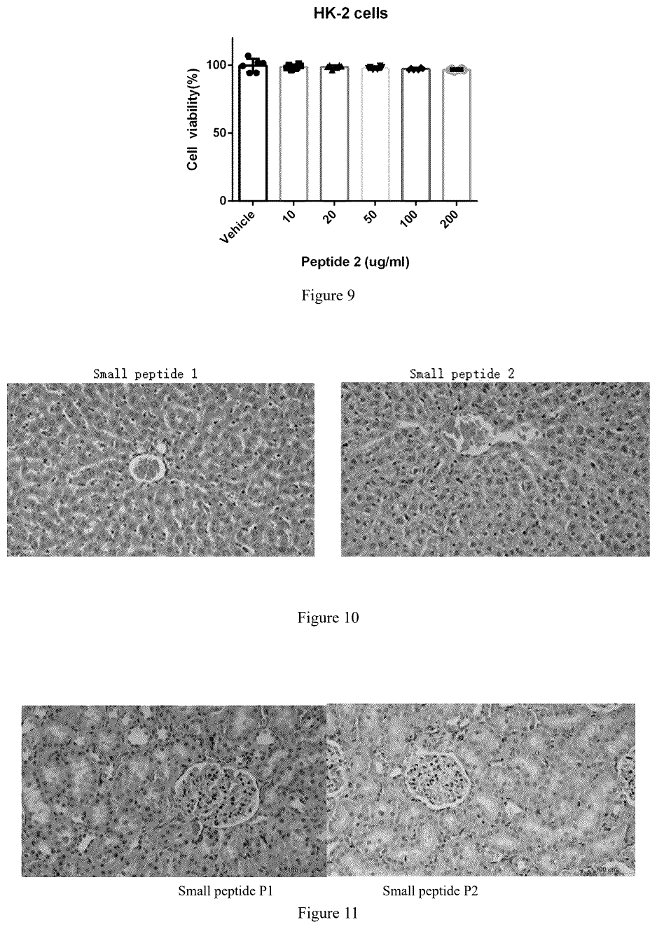

FIG. 9 depicts nephrotoxicity experimental results of the small peptide P2 of Embodiment 5;

FIG. 10 depicts rat liver tissue section images after 14-day administration with 1000 mg/kg of Embodiment 6; and

FIG. 11 depicts rat kidney tissue section images after 14-day administration with 1000 mg/kg of Embodiment 6.

### DETAILED DESCRIPTION

To facilitate understanding of the present disclosure, the following describes the present disclosure in detail with reference to the accompanying drawings. The drawings illustrate preferred embodiments of the present disclosure. However, the present disclosure can be implemented in many various forms and is not limited to the embodiments described herein. The purpose of these embodiments is to provide a thorough and comprehensive understanding of the disclosure of the present disclosure.

Unless otherwise defined, all technical and scientific terms used herein have the same meanings as commonly understood by those one skilled in the art. The terms used in the description of the present disclosure are for the purpose of describing specific embodiments and are not intended to limit the present disclosure. The term “and/or” used herein comprises any and all combinations of one or more related listed items.

Unless otherwise specified, all reagents used in the following embodiments are commercially available, and all methods are conventional.
Rice seed: Polimi-1 (Guangdong Polimi Biotechnology Co., Ltd., Agricultural Plant Variety Protection Application No.: 20181039.9).
Alkaline protease: Purchased from Shanghai Yuanye Bio-Technology Co., Ltd., Product No.: S10154-100 g, packaging specification: 100 g.
Papain: Purchased from Shanghai Yuanye Bio-Technology Co., Ltd., Product No.: S10011-25 g, Specification: 25 g.
Chymotrypsin: Purchased from Shanghai Yuanye Bio-Technology Co., Ltd., Product No.: S10001-10 g, packaging specification: 10 g.
Pepsin: Purchased from Shanghai Yuanye Bio-Technology Co., Ltd., Product No.: S10027-100 g, packaging specification: 100 g.

### Embodiment 1

A small peptide with improved mucosal repair and gastrointestinal function was screened.

### 1. Preparation of an Active Rice Polypeptide Mixture

1) Pre-treatment: 0.2 g of rice seeds were crushed, grinded into powder using liquid nitrogen, and then 1 mL of PBS (phosphate buffer, pH 7.5) was added, and the mixture was homogenized for 15 minutes until a full homogenate was obtained. The homogenate was sonicated in an ice-water bath for 15 minutes to fully release proteins and polypeptides.
2) Enzymatic hydrolysis: A first composite enzyme (comprising alkaline protease and papain at an enzyme activity unit ratio of 5:1) was added to the homogenate at an enzyme activity ratio of 6350 U/g (total enzyme activity units/raw material weight). The enzymatic hydrolysis was performed at pH 8.0 and 37 °C for 25 minutes to obtain a crude enzyme hydrolysate, which was centrifuged at 37 °C and 12,000 rpm for 30 minutes. The supernatant was collected, to perform final enzymatic hydrolysis using a second composite enzyme (comprising chymotrypsin and pepsin at an enzyme activity unit ratio of 4:1) at an enzyme activity ratio of 6350 U/g (total enzyme activity units/raw material weight), at pH 5.0±0.5, and 37±1 °C for 25±0.5 minutes. After centrifugation at 37 °C, 12,000 rpm for 30 minutes, the supernatant, a final enzyme hydrolysate was collected.
3) Purification: The final enzyme hydrolysate was filtered using a 10 kD ultrafiltration tube to collect a filtrate. The filtrate was desalted using conventional methods, filtered, and dried to obtain a rice polypeptide mixture.

### 2. Screening of Active Monomers

The inventors observed the active monomers in the polypeptide mixture of Polimi-1 rice variety by mass spectrometry analysis of the polypeptides from various ordinary rice varieties and Polimi-1 rice variety. The small peptides identical to ordinary rice varieties and with high levels in ordinary rice varieties were excluded by a method of exclusion. Finally, the small peptides with higher levels in Polimi-1 rice variety and lower levels in other ordinary rice varieties were selected for further study, as well as those with higher levels in Polimi-1 rice variety and absent in other ordinary rice varieties. Since peptides with molecular weights below 1500 Daltons were extracted from the mixed polypeptide mixture previously obtained, other peptides above this threshold were excluded. Therefore, multiple candidate small peptides were recognized, and after repeated experiments and screening, the small peptide P1 shown in SEQ ID NO.1 (GASRIIGVDL) and the small peptide P2 shown in SEQ ID NO.2 (GPSGPELRG) were found to have mucosal repair effects.

### Embodiment 2

The small peptide with improved mucosal repair and gastrointestinal function was selected from the small peptide P1 shown in SEQ ID NO.1 (GASRIIGVDL) or the small peptide P2 shown in SEQ ID NO.2 (GPSGPELRG). The small peptides P1 and P2 were prepared using the following method:

I. Synthetic materials and reagents:
1. Protected amino acid raw materials: Fmoc-protected amino acids.
2. Coupling reagents: 2-(1H-Benzotriazole-1-yl)-1,1,3,3-tetramethyluronium hexafluorophosphate (HBTU), N,N-Diisopropylethylamine (DIEA).
3. Solvents: Dimethyl Formamide (DMF), Dichloromethane (DCM), methanol, acetonitrile.
4. Resin: 2-chlorotrityl chloride resin with a substitution degree of 1.1 mmol/g.
5. Deprotection reagent: Piperidine.
6. Detection reagents: Phenol reagent, pyridine reagent, ninhydrin reagent.
7. Cleavage reagents: Trifluoroacetic acid (TFA), Triisopropylsilane (TIS), Ethylenediaminetetraacetic acid (EDT), anhydrous ether.
8. Nitrogen gas.
9. Precision electronic balance.
10. Twelve-channel semi-automatic polypeptide synthesizer.
11. High-performance liquid chromatograph.
12. Freeze dryer.
13. Centrifuge.

### II. Synthetic Process:

1. Resin swelling: 2-chlorotrityl chloride resin was added into a reaction tube, followed by an addition of DMF (15 mL/g), and the mixture was rocked for 60 minutes.
2. Coupling of the first amino acid: The solvent was removed via a sand core filtration, 3 mol of excessive Fmoc-Gly-OH or Fmoc-Leu-OH (the first amino acid at the C-terminus,) and 10 mol of excessive DIEA were added, and the mixture was dissolved in DMF, which was further rocked for 30 minutes. The resin was capped with methanol for 30 minutes.
3. Deprotection: After removing DMF, 20% of piperidine in DMF (15 mL/g) was added to perform 5-minute reaction, and then the piperidine solution was removed. Fresh 20% of piperidine in DMF (15 mL/g) was further added to perform 15-minute reaction.
4. Detection: The piperidine solution was removed again, and a few resin beads were collected, washed three times with ethanol, with addition of ninhydrin, KCN, and phenol solutions (one drop each), and heated at 105 °C to 110 °C for 5 minutes. A positive reaction indicated as dark blue color.
5. Washing: The resin was washed twice with DMF (10 mL/g), twice with methanol (10 mL/g), and twice with DMF (10 mL/g).
6. Coupling: 3 mol of excessive Fmoc-protected amino acid, 3 mol of excessive HBTU, and 10 mol of excessive DIEA were added to the resin, dissolved in DMF, and rocked for 45 minutes.
7. Detection: A few resin beads were collected and washed three times with ethanol, followed by an addition of ninhydrin, pyridine, and phenol solutions (one drop for each), and the mixture was heated at 105 °C to 110 °C for 5 minutes. A negative reaction indicated as a colorless color.
8. Washing: The resin was washed once with DMF (10 mL/g), twice with methanol (10 mL/g), and twice with DMF (10 mL/g).
9. Steps 3 to 8 were repeated to sequentially couple amino acids from right to left in sequence.
10. The resin was further washed twice with DMF (10 mL/g), three times with DCM (10 mL/g), and four times with methanol (10 mL/g), and then dried under vacuum for 10 minutes.
11. Cleavage: A cleavage solution (10 mL/g) of TFA 94.5%, water 2.5%, EDT 2.5% and TIS 1% was prepared for performing a cleavage step for 180 minutes.
12. Drying and washing: The cleaved solution was evaporated with nitrogen gas, precipitated with ether, centrifuged to remove the supernatant, and then the precipitate was washed six times with ether, and air-dried at room temperature.
13. Purification and preparation:
1) A small amount of crude product was dissolved in H₂O/acetonitrile (ACN).
2) A small sample was analyzed via HPLC to determine the retention time of the target peak.
3) A C18 reverse-phase preparative chromatography system was used (Wavelength: 220 nm; Flow rate: 15 mL/min; Injection volume: 20 mL; Column temperature: 25 °C; Buffer A: 0.1% TFA in water; Buffer B: 0.1% TFA in acetonitrile) to collect a target peak solution.
4) A small amount of the target peak solution was taken with a 1.5 mL centrifuge tube for mass spectrometry confirmation and purification.
14. The qualified target peak solution was freeze-dried to obtain the final product.
15. Identification: a small amount of the final product was taken to perform molecular weight identification using Mass spectrometry and purity identification using HPLC.

### III. Characterization of the Small Peptides:

### 1. HPLC Identification:

A small amount of the final product was taken to perform molecular weight identification using Mass spectrometry and purity identification using HPLC.
HPLC identification conditions:
Detection wavelength: 220 nm;
Flow rate: 1.0 mL/min;
Injection volume: 10 μL;
Column: Kromasil 100-5C18, 4.6 mm×250 mm, 5 μm;
Column temperature: 30 °C;
Mobile phase A: 0.1% TFA in acetonitrile;
Mobile phase B: 0.1% TFA in water;

Gradient elution conditions:

| | | A % | | B % | |
|---|---|---|---|---|---|
| 0.0 | min: | 12% | A | 88% | B |
| 20 | min: | 37% | A | 63% | B |
| 20.1 | min: | 100% | A | 0% | B |

The HPLC chromatograms of small peptides P1 and P2 (Peptide 1 and Peptide 2) are shown in FIGS. 1 and 2, respectively. The results indicate that both P1 and P2 exhibit single peaks, confirming the purity of the synthesized peptides.

### 2. Mass Spectrometry Identification

Mass spectrometry identification conditions:
Ion source: ESI Capillary(KV): ±(2500-3500);
Desolvation gas flow rate: 800 L/hr; Desolvation temperature: 450 °C;
Cone voltage: 15-30 V.

The mass spectra of small peptides P1 and P2 are shown in FIGS. 3 and 4, respectively. The results show that the [M+H]⁺ molecular weight of the small peptide P1 is 1000.70, and the [M+2H]²⁺ molecular weight is 500.88, consistent with the theoretical values. The [M+H]⁺ molecular weight of the small peptide P2 is 870.23, and the [M+2H]²⁺ molecular weight is 435.79, also consistent with the theoretical values.

The above experiments demonstrate that the small peptides P1 (GASRIIGVDL) and P2 (GPSGPELRG) shown in SEQ ID NO.1 and SEQ ID NO.2 are successfully synthesized via chemical synthesis.

### Embodiment 3

In this embodiment, the function of the small peptides obtained in Embodiment 2 was validated using a mouse model of acute gastric ulcer.

### 1. Methods

### 1) Grouping:

Control group (model group): A mouse mode of acute gastric ulcer was constructed using anhydrous ethanol (0.1 mL/10 g) (referring to the literature of Study on Action of kangfuxin Solution on Experimental Gastric ulcer [J]. Chinese Traditional Patent Medicine, 2001, 23(2): 122-124. DOI: 10.3969/j.issn.1001-1528.). After modeling, the mice were administered with an equal volume of saline.

Blank group: The mice were administered with an equal volume of saline.

Drug treatment groups: A mouse mode of acute gastric ulcer was constructed by treating the mouse with anhydrous ethanol (0.1 mL/10 g). At 2 h, 12 h, and 22 h after modeling, the mice were administered with the rice polypeptide mixture (50 mg/kg) as the mixture group, small peptide P1 (0.5 mg/kg) as the P1 group, and small peptide P2 (0.5 mg/kg) as the P2 group, respectively.

Each group consisted of 8 mice. After 24 h of modeling, the mice were sacrificed, to extract the gastric tissue, and observe changes in bleeding points and ulcer area via gastric tissue dissection.

### 2. Results

The experimental results were shown in Table 1 and FIG. 5 (gastric tissue section images from the validation experiment related to mouse model of acute gastric ulcer).

TABLE 1
Results of Validation Experiment Related
to Mouse Model of Acute Gastric Ulcer

| | | Ulcer | Ulcer inhibition |
|---|---|---|---|
| Group | Number | index (%) | rate (%) |
| Blank group | 8 | 1.5 ± 0.7 | 0 |
| Control group | 8 | 58.3 ± 10.1 | 0 |
| Polypeptide mixture | 8 | 12.9 ± 7.7 | 78.9% |
| Small peptide P1 | 8 | 8.2 ± 3.1 | 85.9% |
| Small peptide P2 | 8 | 7.1 ± 5.5 | 87.8% |

Note:
The ulcer index was the ratio of ulcer area to gastric mucosal tissue area. The ulcer inhibition rate is the percentage reduction in ulcer area compared to the Control group.

The results indicate that small peptides P1 and P2 exhibit excellent anti-ulcer effects. Compared to the mixture group, P1 group or P2 group in smaller dose (0.5 mg/kg) achieves better results than the mixture in larger dose (50 mg/kg).

### Embodiment 4

The function of the small peptides obtained in Embodiment 2 was validated in the present embodiment by using a rat model of chronic gastric ulcer.

### 1. Methods

### 1) Grouping:

Control group (model group): A rat model of chronic gastric ulcer model was constructed by intragastric administration of anhydrous ethanol (1 mL/200 g), followed by intragastric administration of 200 mg/kg aspirin 2 h later. The rats were administered with aspirin once daily for 4 days. At day 5, the rats were administered with an equal volume of saline.

Blank group: The rats were administered with an equal volume of saline.

Drug treatment groups: A rat model of chronic gastric ulcer was constructed as above-mentioned. At day 5, the rats were respectively administered with the rice polypeptide mixture (50 mg/kg), small peptide P1 (0.5 mg/kg), or small peptide P2 (0.5 mg/kg) twice daily for 3 days. On day 4, the rats were sacrificed and their gastric necropsy was performed.

Positive control group (omeprazole 2.6 mg/kg): A rat model of chronic gastric ulcer was constructed as above. At day 5, the rats were administered with omeprazole (2.6 mg/kg) twice daily for 3 days. On day 4, The rats were sacrificed and their gastric necropsy was performed.

Each group consisted of 10 rats. After 24 h of modeling, the rats were sacrificed to extract the gastric tissue, for measuring gastric pH, and observing changes in ulcer area via gastric tissue dissection. Liver and kidney tissues were collected for pathological section observation to assess drug safety.

### 2. Results

The experimental results were shown in Table 2.

TABLE 2
Results of Validation Experiment Related
to Rat Model of Chronic Gastric Ulcer

| | | Ulcer | Ulcer inhibition |
|---|---|---|---|
| Group | number | index (%) | rate (%) |
| Blank group | 10 | 3 ± 1.1 | 0 |
| Control group | 10 | 40.5 ± 5.3 | 0 |
| Positive control group | 10 | 7.2 ± 4.1 | 82.2% |
| Small peptide P1 | 10 | 5.8 ± 2.5 | 85.7% |
| Small peptide P2 | 10 | 5.5 ± 3.7 | 86.4% |
| Polypeptide mixture | 10 | 6.1 ± 5.2 | 84.9% |

Note:
The ulcer index was the ratio of ulcer area to gastric mucosal tissue area. The ulcer inhibition rate is the percentage reduction in ulcer area compared to the Control group.

The results indicate that small peptides P1 and P2, as well as the Polimi-1 polypeptide mixture, exhibit anti-ulcer effects compared to the Positive control group (P>0.05). The experiments demonstrate that small peptide P1 and small peptide P2 are the active components in the Polimi-1 rice polypeptide mixture responsible for anti-ulcer effects. Moreover, small peptide P1 and small peptide P2 achieve similar or better results at doses less than one-fifth of the positive control, indicating their high efficacy at low doses.

### Embodiment 5

Hepatotoxicity and nephrotoxicity were observed with respect to the small peptides obtained in Embodiment 2.

### 1. Methods

Referring to the method from a literature (Determination of emodin in L-02 cells and cell culture media with liquid chromatography-mass spectrometry: Application to a cellular toxicokinetic study. J Pharm Biomed Anal. 2012; 71:71-78), normal human hepatic parenchymal L-02 cells (obtained from Shanghai Cell Bank) were seeded into 96-well plates and treated with small peptides P1 and P2 at concentrations of 0 μg/mL, 10 μg/mL, 20 μg/mL, 50 μg/mL, 100 μg/mL, and 200 μg/mL. After 24-hour stimulation, the cytotoxicity was assessed by Cell Counting Kit-8 (CCK-8) assay.

Referring to the method from a literature (Sinomenine improve diabetic nephropathy by inhibiting fibrosis and regulating the JAK2/STAT3/SOCS1 pathway in streptozotocin-induced diabetic rats. Life Sci. 2021; 265:118855), normal human kidney-2 cells (HK-02, HK-2) (Shanghai Cell Bank) were seeded into 96-well plates and administered with small peptides P1 and P2 at concentrations of 0 μg/mL, 10 μg/mL, 20 μg/mL, 50 μg/mL, 100 μg/mL, and 200 μg/mL. After 24-hour stimulation, the cytotoxicity was assessed by Cell Counting Kit-8 (CCK-8) assay.

### 2. Results

The results of hepatotoxicity experiment are shown in FIGS. 6 to 7. The results indicate that small peptides P1 and P2 have no effect on the viability of normal human liver cells L-02 and exhibit no hepatotoxicity.

The results of nephrotoxicity experiment are shown in FIGS. 8 to 9. The results indicate that small peptides P1 and P2 have no effect on the viability of normal human kidney cells HK-02 (HK-2) and exhibit no nephrotoxicity.

### Embodiment 6

The small peptides were used for Animal safety observation experiment.

### 1. Methods

The rats were administered with the small peptides via intragastric gavage at doses of 500 mg/kg and 1000 mg/kg daily for 14 days. Changes in body weight were recorded. On day 14, the rats were sacrificed to measure alaninetransaminase (ALT) activity and blood urea nitrogen (BUN) levels in rat serum for assessing whether there was hepatotoxicity and nephrotoxicity in rats.

### 2. Results

The effects of small peptides P1 and P2 (1000 mg/kg) on rat body weight are shown in Tables 3 and 4. Liver and kidney tissue section images of rats after 14-day administration (1000 mg/kg) are shown in FIGS. 10 to 11.

TABLE 3
Effects of Small peptide P1 (1000 mg/kg) on Rat Body Weight

| | Rat 1 | Rat 2 | Rat 3 | Rat 4 | Rat 5 | Rat 6 | Rat7 |
|---|---|---|---|---|---|---|---|
| Time | (g) | (g) | (g) | (g) | (g) | (g) | (g) |
| Day 1 | 210 | 214 | 218 | 217 | 216 | 216 | 213 |
| Day 2 | 212 | 215 | 219 | 220 | 222 | 217 | 215 |
| Day 3 | 212 | 216 | 220 | 221 | 224 | 209 | 218 |
| Day 4 | 214 | 218 | 222 | 224 | 227 | 219 | 220 |
| Day 5 | 215 | 220 | 224 | 228 | 228 | 220 | 225 |
| Day 6 | 215 | 227 | 224 | 230 | 229 | 223 | 228 |
| Day 7 | 216 | 228 | 229 | 236 | 231 | 224 | 229 |
| Day 8 | 218 | 229 | 232 | 235 | 233 | 228 | 231 |
| Day 9 | 220 | 229 | 234 | 238 | 233 | 230 | 234 |
| Day 10 | 225 | 231 | 237 | 239 | 238 | 234 | 236 |
| Day 11 | 226 | 232 | 238 | 241 | 241 | 237 | 239 |
| Day 12 | 227 | 233 | 238 | 243 | 244 | 239 | 243 |
| Day 13 | 228 | 235 | 239 | 244 | 248 | 240 | 244 |
| Day 14 | 230 | 237 | 240 | 247 | 249 | 243 | 243 |
| Day 15 | 235 | 238 | 242 | 248 | 250 | 245 | 245 |

TABLE 4
Effects of Small peptide P2 (1000 mg/kg) on Rat Body Weight

| | Rat 1 | Rat 2 | Rat 3 | Rat 4 | Rat 5 | Rat 6 | Rat7 |
|---|---|---|---|---|---|---|---|
| Time | (g) | (g) | (g) | (g) | (g) | (g) | (g) |
| Day 1 | 208 | 215 | 219 | 218 | 217 | 217 | 214 |
| Day 2 | 210 | 216 | 220 | 221 | 223 | 218 | 216 |
| Day 3 | 211 | 220 | 221 | 222 | 225 | 210 | 219 |
| Day 4 | 213 | 224 | 223 | 225 | 228 | 220 | 221 |
| Day 5 | 214 | 225 | 225 | 229 | 229 | 221 | 226 |
| Day 6 | 214 | 226 | 225 | 231 | 230 | 224 | 229 |
| Day 7 | 215 | 226 | 230 | 237 | 232 | 225 | 230 |
| Day 8 | 216 | 229 | 233 | 236 | 234 | 229 | 232 |
| Day 9 | 221 | 230 | 235 | 239 | 234 | 231 | 235 |
| Day 10 | 224 | 231 | 238 | 240 | 239 | 235 | 237 |
| Day 11 | 225 | 234 | 239 | 242 | 242 | 238 | 240 |
| Day 12 | 230 | 235 | 239 | 244 | 245 | 240 | 244 |
| Day 13 | 231 | 238 | 240 | 245 | 249 | 241 | 245 |
| Day 14 | 235 | 239 | 241 | 248 | 250 | 244 | 244 |
| Day 15 | 238 | 241 | 242 | 250 | 252 | 245 | 245 |

TABLE 5
Effects of Small peptide on Rat Liver Injury (Alaninetransaminase Activity, IU/L)

| Small | | 500 | 1000 | Small | | 500 | 1000 |
|---|---|---|---|---|---|---|---|
| peptide P1 | Saline | mg/kg | mg/kg | peptide P2 | Saline | mg/kg | mg/kg |
| Rat 1 | 50 | 20 | 50 | Rat 1 | 45 | 34 | 45 |
| Rat 2 | 18 | 15 | 27 | Rat 2 | 23 | 17 | 33 |
| Rat 3 | 44 | 21 | 47 | Rat 3 | 43 | 21 | 32 |
| Rat 4 | 32 | 27 | 44 | Rat 4 | 32 | 28 | 38 |
| Rat 5 | 29 | 35 | 32 | Rat 5 | 19 | 34 | 29 |
| Rat 6 | 42 | 44 | 39 | Rat 6 | 23 | 42 | 43 |
| Average | 35.83333 | 27 | 39.83333 | | 30.83333 | 29.33333 | 36.66667 |

TABLE 6
Effects of Small peptide on Rat Kidney Injury
(Blood Urea Nitrogen Levels, mg/dL)

| Small | | 500 | 1000 | Small | | 500 | 1000 |
|---|---|---|---|---|---|---|---|
| peptide P1 | Saline | mg/kg | mg/kg | peptide P2 | Saline | mg/kg | mg/kg |
| Rat 1 | 28 | 33 | 36 | Rat 1 | 24 | 34 | 39 |
| Rat 2 | 31 | 36 | 30 | Rat 2 | 32 | 42 | 43 |
| Rat 3 | 32 | 37 | 34 | Rat 3 | 35 | 33 | 44 |
| Rat 4 | 34 | 25 | 28 | Rat 4 | 39 | 19 | 23 |
| Rat 5 | 33 | 39 | 30 | Rat 5 | 43 | 37 | 29 |
| Rat 6 | 36 | 29 | 32 | Rat 6 | 23 | 32 | 35 |

The above tables show that small peptides P1 and P2 have minimal effects on rat body weight without hepatotoxicity and nephrotoxicity.

The section results indicate: Hepatocytes are arranged regularly, with no obvious edema, fatty degeneration, or necrosis. Hepatic sinusoids show no significant dilation. Minimal inflammatory cell infiltration is observed in the interstitial, with no obvious fibrous tissue hyperplasia. The cortex and medulla of kidney tissue are clearly demarcated, with normal distribution of glomeruli and renal tubules. Glomeruli show no obvious sclerosis. Renal tubular epithelial cells exhibit no obvious degeneration or necrosis. No obvious tubular dilation or casts are observed. Some interstitial congestion is present, with no significant inflammatory cell infiltration or fibrous tissue hyperplasia.

The above embodiments can be combined in any way. For brevity, all possible combinations of the technical features in the embodiments are not described. However, as long as there is no contradiction in the combination of these technical features, they should be considered within the scope of the specification.

The above embodiments express several implementation modes of the present disclosure, which are described specifically and detailly and cannot be understood as a limitation of the patent scope within the present disclosure. It should be pointed out that, for ordinary one skilled in the art, several modifications and improvements can also be made, which are within the protection scope of the present disclosure, without departing from the contents of the present disclosure. Therefore, the protection scope of the present disclosure patent shall be subject to the appended claims.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A small peptide, wherein the small peptide is selected from a basic small peptide shown in SEQ ID NO.1 or SEQ ID NO.2, or a precursor small peptide comprising the basic small peptide, wherein the precursor small peptide is enzymatically hydrolyzed by digestive enzymes in animals to obtain the basic small peptide.

2. A method for preparing the small peptide of claim 1, comprising Fmoc solid-phase synthesis method.

3. The method for preparing the small peptide of claim 2, wherein the method comprises the following steps: covalently coupling the first amino acid at the C-terminus of a small peptide onto a solid-phase vehicle, then performing a synthesis from the N-terminus of this amino acid as a starting point, sequentially removing the amino-protecting group and reacting with an excessive activated second amino acid to extend a peptide chain, repeating above process to obtain the small peptide, cleaving the small peptide from the solid-phase vehicle, and purifying the small peptide to obtain a final product.

4. The method for preparing the small peptide of claim 43, wherein the solid-phase vehicle is 2-chlorotrityl chloride resin.

5. A method of preventing and treating a mucosal injury disease in a mammal in need thereof, comprising administering the small peptide of claim 1.

6. The method of claim 5, wherein the mucosal injury disease comprises oral ulcers, esophageal ulcers, gastric ulcers, intestinal ulcers, and genital mucosal inflammation.

7. The method of claim 6, wherein the gastric ulcers comprise acute gastric ulcers and chronic gastric ulcers.

8. A method of preventing and treating a gastrointestinal disease in a mammal in need thereof, comprising administering the small peptide of claim 1.

9. The method of claim 8, wherein the gastrointestinal disease comprises constipation, diarrhea, and gastric discomfort.

10. The method of claim 9, wherein the gastric discomfort comprises acid reflux, belching, indigestion, stomach bloating, stomach pain, vomiting, and nausea.

11. A pharmaceutical composition, comprising the small peptide of claim 1 and a pharmaceutically acceptable excipient.


SEQUENCE LISTING (2 sequences)

SEQ ID NO: 1 - protein, 10 aa, synthetic construct
GASRIIGVDL

SEQ ID NO: 2 - protein, 9 aa, synthetic construct
GPSGPELRG